(19) United States
(12) Patent Application Publication
(10) Pub. No.: US 2022/0151786 A1
(43) Pub. Date: May 19, 2022

(54) POROUS OFFSET COUPLER

(71) Applicant: Shahan Yacoubian, Burbank, CA (US)

(72) Inventor: Shahan Yacoubian, Burbank, CA (US)

(21) Appl. No.: 17/525,726

(22) Filed: Nov. 12, 2021

Related U.S. Application Data
(60) Provisional application No. 63/114,166, filed on Nov. 16, 2020.

(51) Int. Cl. A61F 2/30; A61F 2/46; A61F 2/38

(52) U.S. Cl. CPC A61F 2/30734; A61F 2/4684; A61F 2/3859; A61F 2/389; A61F 2002/3069; A61F 2002/3093; A61F 2002/30537; A61F 2002/3092; A61F 2310/00017; A61F 2310/00023; A61F 2310/00029; A61F 2310/00131; A61F 2002/30736; A61F 2002/30784

(57) ABSTRACT

An offset coupler is provided. The offset coupler includes a body having a stem coupling component and revision coupling component. The revision coupling component is offset from the stem coupling component along a longitudinal axis. The body includes a porous material which allows bone to grow into the porous material.

### CROSS REFERENCE TO RELATED APPLICATION

The present application claims priority from U.S. Provisional Application No. 63/114,166 filed on Nov. 16, 2020, and titled “POROUS OFFSET COUPLER,” the entire contents of which are incorporated herein by reference for all purposes.

### BACKGROUND

### 1. Field

The present disclosure relates generally to systems and methods related to an offset coupler. In at least one example, the present disclosure relates to an offset coupler with porous metal for use in knee arthroplasty.

### 2. Discussion of Related Art

Conventional revision knee technology in press fit un-cemented total knees utilize offset coupler devices in order to optimize component position when using intramedullary stems. The purpose of offset couplers is to allow for optimal component positioning while allowing for femoral and/or tibial diaphyseal engagement.

### BRIEF SUMMARY

The present inventive concept provides an offset coupler operable to couple revision components, for example in a total knee arthroplasty. The offset coupler includes a revision coupling component that is offset from a stem coupling component along a longitudinal axis. Accordingly, the revision components can coupled with one another while being properly aligned. Additionally, the offset coupler includes a porous material which allows bone to grow into the porous material. By allowing bone to grow into the porous material, the offset coupler is better fixated with the bone such that the revision components are more secure while providing better strength and durability.

The aforementioned may be achieved in an aspect of the present inventive concept by providing an offset coupler. The offset coupler may include a body that has a stem coupling component and a revision coupling component. The revision coupling component may be offset from the stem coupling component along a longitudinal axis. The body may include a porous material which allows bone to grow into the porous material.

The porous material may include stainless steel, cobalt-chromium alloy, titanium, titanium alloy, and/or tantalum. The porous material may include a closed-cell foam. In some examples, the porous material may include an open-cell foam. An adjustable portion may be coupled with the body and include the revision coupling component. The adjustable portion may be operable to be adjusted to move the position of the revision coupling component in relation to the stem coupling component. In at least one example, the adjustable portion may rotate in relation to the body. In some examples, the adjustable portion may slide the revision coupling component along a top surface of the body.

The aforementioned may also be achieved in an aspect of the present inventive concept by providing a system of revision components. The system may include a femoral portion and an offset coupler. The femoral portion may include a femoral stem and a femoral trial. The femoral stem may be operable to be inserted into a femur for disphyseal engagement. The offset coupler may include a body which has a stem coupling component operable to couple with the femoral stem and a revision coupling component operable to couple with the femoral trial. The revision coupling component may be offset from the stem coupling component along a longitudinal axis. The body may include a porous material which allows bone to grow into the porous material.

The aforementioned may also be achieved in an aspect of the present inventive concept by providing a system of revision components. The system may include a tibial portion and an offset coupler. The tibial portion may include a tibial stem and a tibial tray. The tibial stem may be operable to be inserted into a tibia for disphyseal engagement. The offset coupler may include a body which has a stem coupling component operable to couple with the tibial stem and a revision coupling component operable to couple with the tibial tray. The revision coupling component may be offset from the stem coupling component along a longitudinal axis. The body may include a porous material which allows bone to grow into the porous material.

The foregoing is intended to be illustrative and is not meant in a limiting sense. Many features of the embodiments may be employed with or without reference to other features of any of the embodiments. Additional aspects, advantages, and/or utilities of the present inventive concept will be set forth in part in the description that follows and, in part, will be apparent from the description, or may be learned by practice of the present inventive concept.

### BRIEF DESCRIPTION OF THE DRAWINGS

The description will be more fully understood with reference to the following figures and data graphs, which are presented as various embodiments of the present inventive concept and should not be construed as a complete recitation of the scope of the present inventive concept, wherein:

FIG. 1A illustrates an example of a revision knee in accordance with the present disclosure;

FIG. 1B illustrates an exploded, side view of an example of revision components;

FIG. 10 illustrates an exploded, front view of the revision components of FIG. 1B;

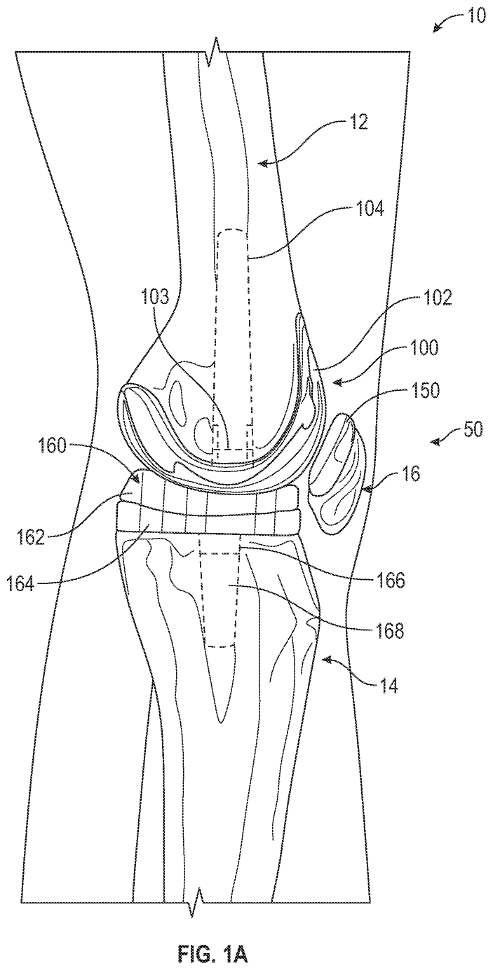

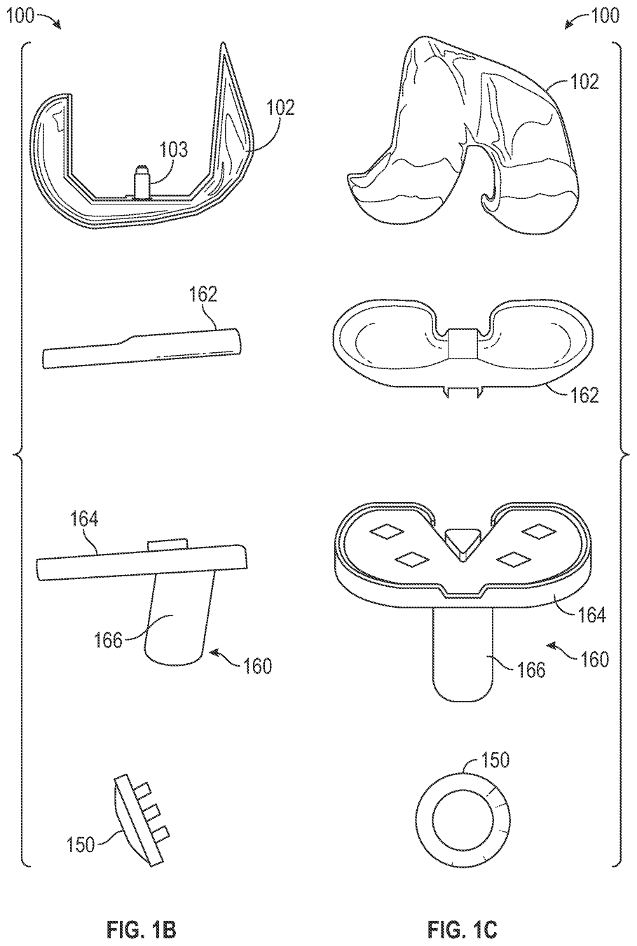

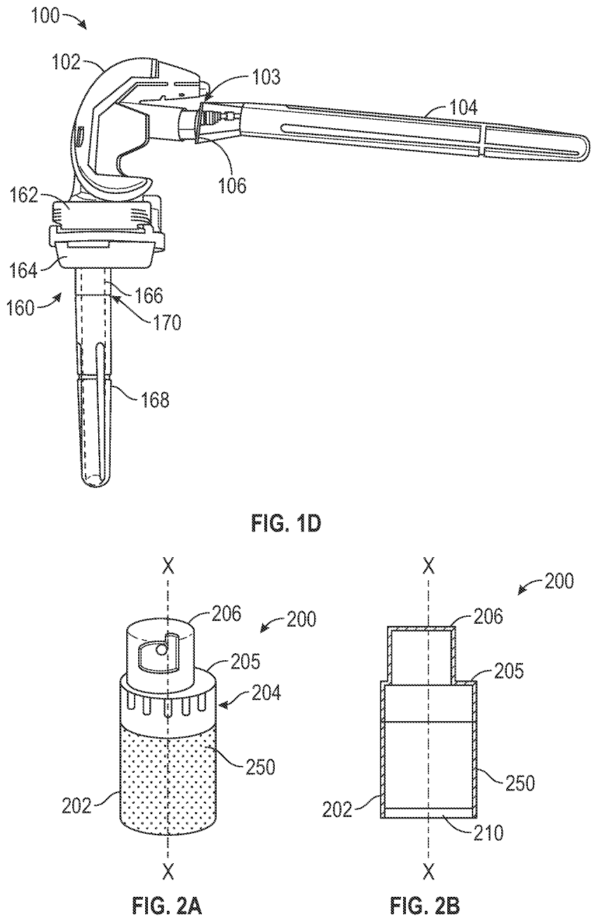

FIG. 1D illustrates an example of assembled revision components;

FIG. 2A illustrates an example of an offset coupler;

FIG. 2B illustrates a cross-sectional view of an offset coupler;

FIG. 3 illustrates an exploded view of femoral portion of revision components with an offset coupler; and

FIG. 4 illustrates an example of assembled revision components with an offset coupler utilized in tibial portion of revision components.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims. Further, it should be understood that any one of the features of the present inventive concept may be used separately or in combination with other features. Other systems, methods, features, and advantages of the present inventive concept will be, or become, apparent to one with skill in the art upon examination of the figures and the detailed description. It is intended that all such additional systems, methods, features, and advantages be included within this description, be within the scope of the present inventive concept, and be protected by the accompanying claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described. The term “real-time” or “real time” means substantially instantaneously.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

### II. General Architecture

The disclosure now turns to FIG. 1A, which illustrates an exemplary revision knee 10, in which the present disclosure may be implemented. The revision knee 10 includes a femur 12, a tibia 14, and a patella 16. Revision components 50 can be inserted and/or coupled with any combination of the femur 12, tibia 14, and/or patella 16 as needed. A total revision knee arthroplasty, as shown in FIG. 1A, includes revision to each of the femur 12, tibia 14, and patella 16.

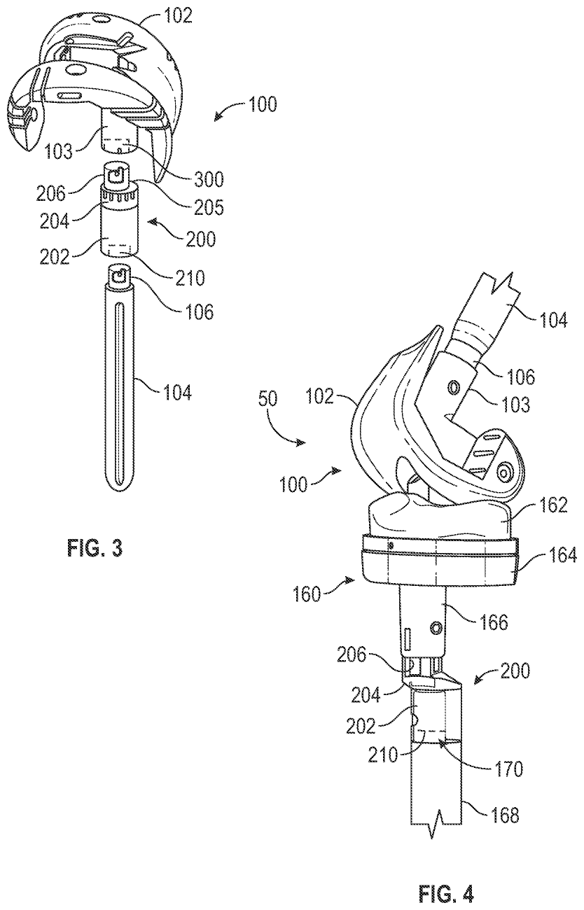

Referring also to FIGS. 1B, 10, and 1D, revision components 50 can include a femoral portion 100, a tibial portion 150, and/or a patella portion 150.

The femoral portion 100 can include a femoral stem 104 and a femoral trial 102. The femoral stem 104 is operable to be inserted into the femur 12 for disphyseal engagement with the bone. In at least one example, to insert the femoral stem 104 into the femur 12, a canal can be reamed into the shaft of the femur 12 along a longitudinal axis. The femoral stem 104 is inserted into the reamed canal in the femur 12.

In at least one example, to promote bony contact of the femoral portion 100 with the femur 12, at least a portion of the femoral stem 104 and/or at least a portion of the femoral trial 102 that is in contact with the femur 12 can be made of a porous material. The porous material allows bone to grow into the desired portions of the femoral stem 104 and/or the femoral trial 102 to achieve fixation of the revision components 50 with the femur 12. In some examples, the porous material can include stainless steel, cobalt-chromium alloy, titanium, titanium alloy, and/or tantalum. Other suitable materials can be used for the femoral portion 100 without deviating from the scope of the present disclosure so long as the materials are bio-compatible and have sufficient strength and durability.

The femoral trial 102 can be coupled with the femoral stem 104 and cover at least a portion of the end of the femur 12. The femoral trial 102 includes a trial coupling component 103, and the femoral stem 104 includes a stem coupling component 106. In some examples, the femoral trial 102 can be directly coupled with the femoral stem 104 by coupling the trial coupling component 103 with the stem coupling component 106. In at least one example, the trial coupling component 103 and/or the stem coupling component 106 can be coupled by morse taper, threaded engagement, adhesive, and/or any other suitable coupling mechanism without deviating from the scope of the present disclosure. In at least one example, the stem coupling component 106 can be inserted into the trial coupling component 103. In some examples, the trial coupling component 103 can be inserted into the stem coupling component 106.

The tibial portion 160 includes a spacer 162, a tibial tray 164 operable to receive the spacer 162, and a tibial stem 168. The spacer 162 is operable to receive the femoral trial 102 and provide support for the body as the knee 10 flexes and bends. In at least one example, the spacer 162 can be made of a medical-grade and bio-compatible material. In some examples, the spacer 162 can be made of a medical-grade plastic. In some examples, the spacer 162 can be made of polyethylene. Other materials for the spacer 162 can be utilized without deviating from the scope of the present disclosure.

The tibial stem 168 is operable to be inserted into the tibia 14 for disphyseal engagement with the bone. In at least one example, to insert the tibial stem 168 into the tibia 14, a canal can be reamed into the shaft of the tibia 14 along a longitudinal axis. The tibial stem 168 is inserted into the reamed canal in the tibia 14.

In at least one example, to promote bony contact of the tibial portion 160 with the tibia 14, at least a portion of the tibial stem 168 and/or at least a portion of the tibial tray 164 that is in contact with the tibia 14 can be made of a porous material. The porous material allows bone to grow into the desired portions of the femoral stem 104 and/or the tibial tray 164 to achieve fixation of the revision components 50 with the tibia 14. In some examples, the porous material can include stainless steel, cobalt-chromium alloy, titanium, titanium alloy, and/or tantalum. Other suitable materials can be used for the tibial portion 160 without deviating from the scope of the present disclosure so long as the materials are bio-compatible and have sufficient strength and durability.

The tibial tray 164 can be coupled with the tibial stem 168 and cover at least a portion of the end of the tibia 14. The tibial tray 164 includes a tray coupling component 166, and the tibial stem 168 includes a stem coupling component 170. In some examples, the tibial tray 164 can be directly coupled with the tibial stem 168 by coupling the tray coupling component 166 with the stem coupling component 170. In at least one example, the tray coupling component 166 and/or the stem coupling component 170 can be coupled by morse taper, threaded engagement, adhesive, and/or any other suitable coupling mechanism without deviating from the scope of the present disclosure. In at least one example, the stem coupling component 170 can be inserted into the tray coupling component 166. In some examples, the tray coupling component 166 can be inserted into the stem coupling component 170.

The patella portion 150 can be coupled with the patella 16 to provide the proper shape and fit of the patella 16 to the revision knee 10. In at least one example, the patella portion 150 can be made of plastic. The material of the patella portion 150 can be any suitable material without deviating from the scope of the present disclosure.

To optimize bony contact of the revision components 50, an offset coupler 200 as illustrated in FIGS. 2A and 2B, can be used to couple the revision components 50. The offset coupler 200 allows both the revision components 50 to be in optimal position and coupled to each other. Without an offset coupler 200, optimal positioning of the revision components 50 can be compromised. While the present disclosure utilizes revision knee arthroplasty, the offset coupler 200 as disclosed herein can be utilized for other procedures to promote optimal positioning of components.

The offset coupler 200 can include a body 202 having a stem coupling component 210 and a revision coupling component 206. In at least one example, as shown in FIG. 2B, the stem coupling component 210 can be opposite the revision coupling component 206 in relation to the body 202. The stem coupling component 210 is operable to couple with a stem revision component, for example a femoral stem 104 or a tibial stem 168. The revision coupling component 206 is operable to couple with a revision component 50, for example a femoral trial 102 or a tibial tray 164. In at least one example, the stem coupling component 210 and/or the revision coupling component 206 can be coupled with the revision components 50 by morse taper, threaded engagement, adhesive, and/or any other suitable coupling mechanism without deviating from the scope of the present disclosure.

For example, as illustrated in FIG. 3, the offset coupler 200 is utilized in the femoral portion 100 of the revision components 50. The stem coupling component 210 is coupled with the femoral stem 104 by interacting with the stem coupling component 106. As illustrated in FIG. 3, the stem coupling component 106 which projects from the femoral stem 104 is inserted into the stem coupling component 210 which is formed as a recess. In other examples, the stem coupling component 106 can form a recess and can receive the stem coupling component 210 which projects from the body 202 of the offset coupler 200. In at least one example, the stem coupling component 210 can be coupled with the femoral stem 104 by morse taper, threaded engagement, adhesive, and/or any other suitable coupling mechanism without deviating from the scope of the present disclosure.

The revision coupling component 206 is coupled with the femoral trial 102 by interacting with the trial coupling component 103. As illustrated in FIG. 3, the revision coupling component 206 which projects from the body 202 of the offset coupler 200 is inserted into the trial coupling component 103 which is formed as a recess. In other examples, the revision coupling component 206 can form a recess and can receive the trial coupling component 103 which projects from the femoral trial 102. In at least one example, the revision coupling component 206 can be coupled with the femoral trial 102 by morse taper, threaded engagement, adhesive, and/or any other suitable coupling mechanism without deviating from the scope of the present disclosure.

As another example, as illustrated in FIG. 4, the offset coupler 200 is utilized in the tibial portion 160 of the revision components 50. The stem coupling component 210 is coupled with the tibial stem 168 by interacting with the stem coupling component 170. As illustrated in FIG. 4, the stem coupling component 170 which projects from the tibial stem 168 is inserted into the stem coupling component 210 which is formed as a recess. In other examples, the stem coupling component 170 can form a recess and can receive the stem coupling component 210 which projects from the body 202 of the offset coupler 200. In at least one example, the stem coupling component 210 can be coupled with the tibial stem 168 by morse taper, threaded engagement, adhesive, and/or any other suitable coupling mechanism without deviating from the scope of the present disclosure.

The revision coupling component 206 is coupled with the tibial tray 164 by interacting with the tray coupling component 166. As illustrated in FIG. 4, the revision coupling component 206 which projects from the body 202 of the offset coupler 200 is inserted into the tray coupling component 166 which is formed as a recess. In other examples, the revision coupling component 206 can form a recess and can receive the tray coupling component 166 which projects from the tibial tray 164. In at least one example, the revision coupling component 206 can be coupled with the tibial tray 164 by morse taper, threaded engagement, adhesive, and/or any other suitable coupling mechanism without deviating from the scope of the present disclosure.

FIGS. 3 and 4 illustrate the offset coupler 200 being a separate component from the revision components 50 (for example the femoral stem 104, the femoral trial 102, the tibial stem 168, and/or the tibial tray 164). Accordingly, the offset coupler 200 can be utilized only when needed. In some examples, the offset coupler 200 may be integrated with the revision components 50. For example, the offset coupler 200 may be integrated with any combination of the femoral stem 104, the femoral trial 102, the tibial stem 168, and/or the tibial tray 164. With the integration of the offset coupler 200 in the revision components 50, flexibility to ensure proper alignment is provided while enhancing the mechanical strength by removing an extra point of connection.

The offset coupler 200 provides optimal positioning of the revision components 50 by allowing correction of misalignment of the revision components 50. To correct any misalignment, the revision coupling component 206 is offset from the stem coupling component 210 along a longitudinal axis X-X.

In at least one example, the body 202 includes an adjustable portion 204 coupled with the body 202. The adjustable portion 204 can include the revision coupling component 206. The adjustable portion 204 can be operable to be adjusted to move the position of the revision coupling component 206 in relation to the stem coupling component 210. The offset coupler 200 can then provide varying degrees of offset between the revision coupling component 206 and the stem coupling component 210. For example, as illustrated in FIG. 2A, the adjustable portion 204 may rotate in relation to the body 202. In some examples, the adjustable portion 204 may slide the revision coupling component 206 along a top surface 205 of the body 202. The top surface 205 of the body 202 can be referred to as the surface of the body 202 which includes the revision coupling component 206. When the revision coupling component 206 is in a desired position, the adjustable portion 204 may be locked to prevent further movement of the revision coupling component 206. Other suitable methods and mechanisms to move the revision coupling component 206 in relation to the stem coupling component 210 can be utilized without deviating from the scope of the present disclosure.

In at least one example, a porous material 250 can be used during revision knee surgery when there is significant bone loss and that bone loss need to addressed in order to achieve stable fixation of the implant. At least a portion of the offset coupler 200 includes the porous material 250 which allows bone to grow into the porous material 250, promoting fixation of the offset coupler 200 with the bone. As illustrated in FIGS. 2A and 2B, the body 202 of the offset coupler 200 can include the porous material 250.

In at least one example, the porous material 250 can include a closed-cell foam. In closed-cell foams, each cell is completely enclosed by a thin wall or membrane of metal. In some examples, the porous material 250 can include an open-cell foam. In open-cell foams the individual cells are interconnected, allowing tissue to infiltrate the foam and anchor the offset coupler 200 into position.

In at least one example, the porous material 250 can include partly or fully porous-coated solid substrates. In some examples, the porous material 250 can include fully porous materials. In some examples, the porous material 250 can include a porous segment joined to a solid part.

In at least one example, the porous material 250 can include stainless steel, cobalt-chromium alloy, titanium, titanium alloy, and/or tantalum. Other suitable materials can be utilized without deviating from the scope of the disclosure so long as the material provides adequate biocompatibility, strength, and durability to be used in knee arthroplasty.

In determining biocompatibility, the porous material 250 supports normal cellular activity without any local and systemic toxic effects to the host tissue. The porous material 250 can be osteoconductive and/or osteoinductive and be able to induce blood vessels formation within or around the offset coupler 200. Additionally, the porous material 250 can be non-immunogenic.

In determining mechanical properties, the porous material 250 provides mechanical properties similar to the host bone with a sufficient mechanical strength. The porous material 250 can form a matrix that possesses mechanical properties that are similar to the tissue in the immediate surrounding area of the defect. Accordingly, the bone can sufficiently grow into the porous material 250 while providing sufficient mechanical support and fixation.

In determining pore size, the porous material 250 can have pores that have macro-(pore size >100 mm) and micro-porosity (pore size <20 mm), and the pores can be interconnected. However, the porosity can reduce mechanical properties such as compressive strength and resistance to corrosion. Accordingly, the amount of pores and pore size is determined such that sufficient mechanical support, durability, and fixation to the bone is maintained.

As illustrated in FIGS. 2A and 2B, the porous material 250 is provided on the external walls of the body 202. In some examples, the porous material 250 may be formed throughout the entire body 202 of the offset coupler 200. The offset coupler 200, by allowing bone to grow into the porous material 250, accomplishes bone fixation and integration of the offset coupler 200 with the bone (for example femur 12 or tibia 14) in order to achieve long term fixation and ultimate success. For the long term success of cementless implants, bone ingrowth around and/or with the porous material 250 of the offset coupler 200 is crucial.

The application of the offset coupler 200 provides a varying range of “offset-ability” which provides proper alignment and placement of revision components 50 in combination with the porous material 250 which allows for direct engagement and contact of the revision components 50 and/or the offset coupler 200 with metaphyseal bone to promote bone integration. By combining these features into one utilitarian construct, the offset coupler 200 achieves fixation during revision knee arthroplasty while optimizing revision component 50 placement to address intra operative clinical needs.

The embodiments shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the embodiments described above may be modified within the scope of the appended claims.

What is claimed is:

1. An offset coupler comprising:
a body having a stem coupling component and a revision coupling component, wherein the revision coupling component is offset from the stem coupling component along a longitudinal axis,
wherein the body includes a porous material which allows bone to grow into the porous material.

2. The offset coupler of claim 1, wherein the porous material includes stainless steel, cobalt-chromium alloy, titanium, titanium alloy, and/or tantalum.

3. The offset coupler of claim 1, wherein the porous material includes a closed-cell foam.

4. The offset coupler of claim 1, wherein the porous material includes an open-cell foam.

5. The offset coupler of claim 1, further comprising an adjustable portion coupled with the body including the revision coupling component, the adjustable portion operable to be adjusted to move the position of the revision coupling component in relation to the stem coupling component.

6. The offset coupler of claim 5, wherein the adjustable portion rotates in relation to the body.

7. The offset coupler of claim 5, wherein the adjustable portion slides the revision coupling component along a top surface of the body.

8. A system of revision components comprising:
a femoral portion including:
a femoral stem operable to be inserted into a femur for disphyseal engagement; and
a femoral trial; and
an offset coupler including a body, the body having:
a stem coupling component operable to couple with the femoral stem; and
a revision coupling component operable to couple with the femoral trial,
wherein the revision coupling component is offset from the stem coupling component along a longitudinal axis,
wherein the body includes a porous material which allows bone to grow into the porous material.

9. The system of claim 8, wherein the porous material includes stainless steel, cobalt-chromium alloy, titanium, titanium alloy, and/or tantalum.

10. The system of claim 8, wherein the porous material includes a closed-cell foam.

11. The system of claim 8, wherein the porous material includes an open-cell foam.

12. The system of claim 8, further comprising an adjustable portion coupled with the body including the revision coupling component, the adjustable portion operable to be adjusted to move the position of the revision coupling component in relation to the stem coupling component.

13. The system of claim 12, wherein the adjustable portion rotates in relation to the body.

14. The system of claim 12, wherein the adjustable portion slides the revision coupling component along a top surface of the body.

15. A system of revision components comprising:
a tibial portion including:
a tibial stem operable to be inserted into a tibia for disphyseal engagement; and
a tibial tray; and
an offset coupler including a body, the body having:
a stem coupling component operable to couple with the tibial stem; and
a revision coupling component operable to couple with the tibial tray,
wherein the revision coupling component is offset from the stem coupling component along a longitudinal axis,
wherein the body includes a porous material which allows bone to grow into the porous material.

16. The system of claim 15, wherein the porous material includes stainless steel, cobalt-chromium alloy, titanium, titanium alloy, and/or tantalum.

17. The system of claim 15, wherein the porous material includes a closed-cell foam.

18. The system of claim 15, wherein the porous material includes an open-cell foam.

19. The system of claim 15, further comprising an adjustable portion coupled with the body including the revision coupling component, the adjustable portion operable to be adjusted to move the position of the revision coupling component in relation to the stem coupling component.

20. The system of claim 15, wherein the adjustable portion rotates in relation to the body.

21. The system of claim 15, wherein the adjustable portion slides the revision coupling component along a top surface of the body.
